(19) United States
(12) Patent Application Publication
(10) Pub. No.: US 2022/0062061 A1
(43) Pub. Date: Mar. 3, 2022

(54) NEGATIVE PRESSURE WOUND THERAPY APPARATUS

(71) Applicant: T.J.Smith and Nephew,Limited, Hull (GB)

(72) Inventors: Benjamin James Gardner, Hull (GB); Samuel John Mortimer, Kingston upon Hull (GB); Amy Nicole Wheldrake, Goole (GB)

(21) Appl. No.: 17/419,193

(22) PCT Filed: Dec. 13, 2019
(86) PCT No.: PCT/EP2019/085089

§ 371 Date: Jun. 28, 2021

(30) Foreign Application Priority Data
Jan. 2, 2019 (GB) 1900015.7

(51) Int. Cl. A61F 13/02

(52) U.S. Cl. CPC A61F 13/0216; A61F 13/0209

(57) ABSTRACT

Disclosed embodiments relate to apparatuses and methods for wound treatment. In certain embodiments, a wound treatment apparatus can comprise a wound dressing configured to be positioned over a wound site. The wound dressing can comprise an absorbent layer for absorbing wound exudate. The absorbent layer can comprise a superabsorbent material, wherein the superabsorbent material is positioned at one or more target areas of the absorbent layer. A backing layer can be provided over the absorbent layer and comprising at least one orifice.

### BACKGROUND

### Technical Field

Embodiments described herein relate to apparatuses, systems, and methods the treatment of wounds, for example using dressings in combination with negative pressure wound therapy.

### Description of the Related Art

The treatment of open or chronic wounds that are too large to spontaneously close or otherwise fail to heal by means of applying negative pressure to the site of the wound is well known in the art. Negative pressure wound therapy (NPWT) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

Many different types of wound dressings are known for aiding in NPWT systems. These different types of wound dressings include many different types of materials and layers, for example, gauze, pads, foam pads or multi-layer wound dressings. One example of a multi-layer wound dressing is the PICO dressing, available from Smith & Nephew, which includes a superabsorbent layer beneath a backing layer to provide a canister-less system for treating a wound with NPWT. The wound dressing may be sealed to a suction port providing connection to a length of tubing, which may be used to pump fluid out of the dressing and/or to transmit negative pressure from a pump to the wound dressing.

Wound dressings for use in negative pressure can have a lifetime of the wound dressing associated with the liquid absorbency capacity of the dressing. The shortened lifetime can be observed due to problems of the fluid pathway to the port being blocked before the dressing is at full absorbent capacity. It may be desirable, in some situations, to provide a fluid flow pathway that prevents or decreases the blocking of the port until the full lifetime of the dressing is achieved.

### SUMMARY

In some cases, a wound treatment apparatus can comprise a wound dressing configured to be positioned over a wound site, the wound dressing can comprise an absorbent layer for absorbing wound exudate, the absorbent layer comprising a superabsorbent material, wherein the superabsorbent material is positioned at one or more target areas of the absorbent layer, a backing layer over the absorbent layer and comprising at least one orifice.

The wound dressing of any preceding paragraphs and/or any of the wound dressings disclosed herein can include one or more of the following features. The one or more target areas can be positioned to allow an air path within the absorbent layer between or around the superabsorbent material in the one or more target areas when the superabsorbent materials are wet. The superabsorbent material can be positioned within the absorbent layer such that when exudate is absorbed by the superabsorbent material, pores within the absorbent layer are opened to allow for flow of air. The superabsorbent material can be screen printed to position the superabsorbent material at the one or more target areas of the absorbent layer. The superabsorbent material can be glued to the absorbent layer to position the superabsorbent material at the one or more target areas of the absorbent layer. The absorbent layer can be a 3D structure, wherein the superabsorbent material is injected into the 3D structure to position the superabsorbent material at the one or more target areas. The absorbent layer can comprise a first nonwoven layer and the superabsorbent material can comprise a second nonwoven layer of superabsorbent fibers, wherein the superabsorbent fibers are bonded onto the first nonwoven to position the superabsorbent material at the one or more target areas. The superabsorbent material can comprise a web of superabsorbent fibers, wherein the superabsorbent fibers are stitch bonded, wherein the stitch bonding of the superabsorbent fibers is configured to restrict the ability of the superabsorbent fibers from swelling when wetted and create air panels in the absorbent layer. The superabsorbent material can comprise a superabsorbent yarn, wherein the superabsorbent yarn is integrated into the absorbent layer. The absorbent layer can comprise a nonwoven web and the superabsorbent yarn is integrated into the nonwoven web. The wound treatment apparatus can further comprise a first transmission layer beneath the absorbent layer. The absorbent layer can comprise a plurality of target areas of superabsorbent material. The plurality of target areas of superabsorbent material can be arranged in a regularly repeating pattern. The plurality of target areas of superabsorbent material can be arranged in a grid pattern. The plurality of target areas of superabsorbent material can comprise a plurality of aggregated superabsorbent particles, each aggregation being spaced apart from one another. The wound treatment apparatus can further comprise a wound contact layer beneath the absorbent layer and sealed to the backing layer. The wound treatment apparatus can further comprise a wound contact layer beneath the absorbent layer and sealed to the backing layer, and without providing a separate transmission layer between the wound contact layer and the backing layer. The wound treatment apparatus can further comprise a source of negative pressure configured to be in fluid communication with a wound site through the wound dressing. A fluidic connector can be positioned over the at least one orifice configured to provide negative pressure through the wound dressing to the wound site. The fluidic connector can comprise a filter.

### BRIEF DESCRIPTION OF THE DRAWINGS

Embodiments of the present disclosure will now be described hereinafter, by way of example only, with reference to the accompanying drawings in which:

FIG. 1A illustrates an embodiment of a negative pressure wound treatment system employing a flexible fluidic connector and a wound dressing capable of absorbing and storing wound exudate;

FIG. 1B illustrates an embodiment of a negative pressure wound treatment system employing a flexible fluidic connector and a wound dressing capable of absorbing and storing wound exudate;

FIG. 2A illustrates an embodiment of a negative pressure wound treatment system employing a flexible fluidic connector and a wound dressing capable of absorbing and storing wound exudate;

FIG. 2B illustrates a cross section of an embodiment of a fluidic connector connected to a wound dressing;

FIGS. 3A-D illustrate the use and application of an embodiment of a wound treatment system onto a patient; and

FIGS. 4A-5B illustrate embodiments of superabsorbent particles within a dressing layer.

### DETAILED DESCRIPTION

Embodiments disclosed herein relate to apparatuses and methods of treating a wound with reduced pressure, including a source of negative pressure and wound dressing components and apparatuses. The apparatuses and components comprising the wound overlay and packing materials, if any, are sometimes collectively referred to herein as dressings.

Preferred embodiments disclosed herein relate to wound therapy for a human or animal body. Therefore, any reference to a wound herein can refer to a wound on a human or animal body, and any reference to a body herein can refer to a human or animal body. The term “wound” as used herein, in addition to having its broad ordinary meaning, includes any body part of a patient that may be treated using negative pressure. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Treatment of such wounds can be performed using negative pressure wound therapy, wherein a reduced or negative pressure can be applied to the wound to facilitate and promote healing of the wound. It will also be appreciated that the wound dressing and methods as disclosed herein may be applied to other parts of the body, and are not necessarily limited to treatment of wounds.

It will be understood that embodiments of the present disclosure are generally applicable to use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist in the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

The negative pressure range for some embodiments of the present disclosure can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some embodiments, the pressure range can be between about −40 mmHg and −150 mmHg. Alternatively, a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even −150 mmHg, can be supplied by the negative pressure apparatus.

In some embodiments of wound closure devices described herein, increased wound contraction can lead to increased tissue expansion in the surrounding wound tissue. This effect may be increased by varying the force applied to the tissue, for example by varying the negative pressure applied to the wound over time, possibly in conjunction with increased tensile forces applied to the wound via embodiments of the wound closure devices. In some embodiments, negative pressure may be varied over time for example using a sinusoidal wave, square wave, and/or in synchronization with one or more patient physiological indices (e.g., heartbeat). Examples of such applications where additional disclosure relating to the preceding may be found in U.S. Pat. No. 8,235,955, titled “Wound treatment apparatus and method,” issued on Aug. 7, 2012; and U.S. Pat. No. 7,753,894, titled “Wound cleansing apparatus with stress,” issued Jul. 13, 2010. The disclosures of both of these patents are hereby incorporated by reference in their entirety.

Embodiments of the wound dressings, wound dressing components, wound treatment apparatuses and methods described herein may also be used in combination or in addition to those described in International Application No. PCT/IB2013/001469, filed May 22, 2013, published as WO 2013/175306 A2 on Nov. 28, 2013, titled “APPARATUSES AND METHODS FOR NEGATIVE PRESSURE WOUND THERAPY,” U.S. patent application Ser. No. 14/418,874, filed Jan. 30, 2015, published as US 2015/0190286 A1 on Jul. 9, 2015, titled “WOUND DRESSING AND METHOD OF TREATMENT,” the disclosures of which are hereby incorporated by reference in their entireties. Embodiments of the wound dressings, wound treatment apparatuses and methods described herein may also be used in combination or in addition to those described in U.S. patent application Ser. No. 13/092,042, filed Apr. 21 2011, published as US2011/0282309, titled “WOUND DRESSING AND METHOD OF USE,” and U.S. patent application Ser. No. 14/715,527, filed May 18, 2015, published as US2016/0339158, titled “FLUIDIC CONNECTOR FOR NEGATIVE PRESSURE WOUND THERAPY,” the disclosures of which are hereby incorporated by reference in their entireties, including further details relating to embodiments of wound dressings, the wound dressing components and principles, and the materials used for the wound dressings. Embodiments of the wound dressings, wound treatment apparatuses and methods described herein may also be used in combination or in addition to those described in International Application No. PCT/EP2018/066570, filed Jun. 21, 2018, titled “NEGATIVE PRESSURE WOUND THERAPY APPARATUS,” the disclosure of which is hereby incorporated by reference in its entirety, including further details relating to embodiments of wound dressings, the wound dressing components and principles, and the materials used for the wound dressings.

Additionally, some embodiments related to TNP wound treatment comprising a wound dressing in combination with a pump and/or associated electronics described herein may also be used in combination or in addition to those described in International Application No. PCT/EP2016/059329, filed Apr. 26, 2016, published as WO2016174048 A1 on Nov. 3, 2016, titled “REDUCED PRESSURE APPARATUS AND METHODS.”

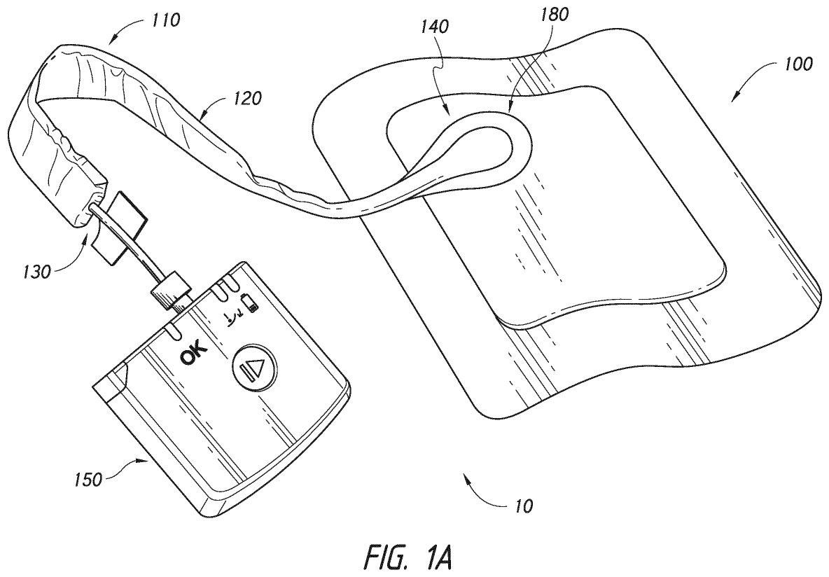

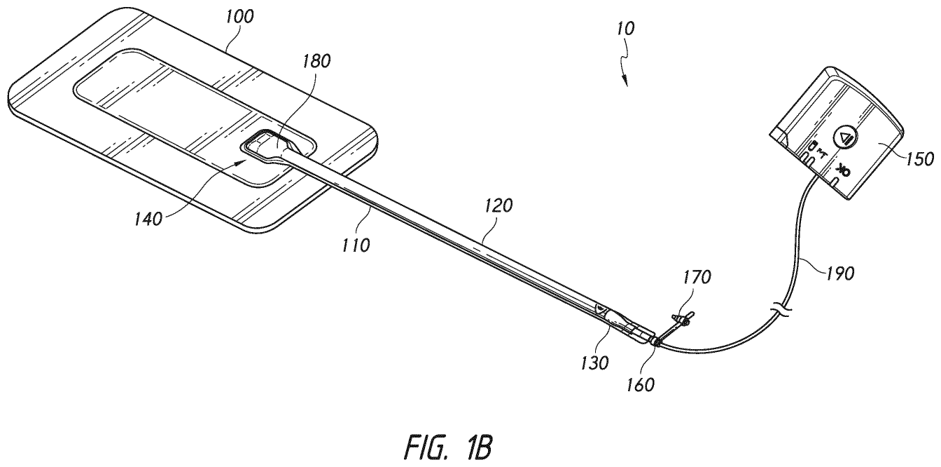

FIGS. 1A-B illustrate embodiments of a negative pressure wound treatment system 10 employing a wound dressing 100 in conjunction with a fluidic connector 110. Here, the fluidic connector 110 may comprise an elongate conduit, more preferably a bridge 120 having a proximal end 130 and a distal end 140, and an applicator 180 at the distal end 140 of the bridge 120. An optional coupling 160 is preferably disposed at the proximal end 130 of the bridge 120. A cap 170 may be provided with the system (and can in some cases, as illustrated, be attached to the coupling 160). The cap 170 can be useful in preventing fluids from leaking out of the proximal end 130. The system 10 may include a source of negative pressure such as a pump or negative pressure unit 150 capable of supplying negative pressure. The pump may comprise a canister or other container for the storage of wound exudates and other fluids that may be removed from the wound. A canister or container may also be provided separate from the pump. In some embodiments, such as illustrated in FIGS. 1A-1B, the pump 150 can be a canisterless pump such as the PICO™ pump, as sold by Smith & Nephew. The pump 150 may be connected to the coupling 160 via a tube 190, or the pump 150 may be connected directly to the coupling 160 or directly to the bridge 120. In use, the dressing 100 is placed over a suitably-prepared wound, which may in some cases be filled with a wound packing material such as foam or gauze. The applicator 180 of the fluidic connector 110 has a sealing surface that is placed over an aperture in the dressing 100 and is sealed to the top surface of the dressing 100. Either before, during, or after connection of the fluidic connector 110 to the dressing 100, the pump 150 is connected via the tube 190 to the coupling 160, or is connected directly to the coupling 160 or to the bridge 120. The pump is then activated, thereby supplying negative pressure to the wound. Application of negative pressure may be applied until a desired level of healing of the wound is achieved. In some embodiments, the pump can be miniaturized and portable, although larger conventional pumps may also be used with the dressing 100. In some embodiments, the pump may be attached or mounted onto or adjacent the dressing 100.

In some embodiments, a source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The wound dressing can include a cover layer for positioning over the layers of the wound dressing. The cover layer can be the upper most layer of the dressing. In some embodiments, the wound dressing can include a second cover layer for positioning over the layers of the wound dressing and any of the integrated components. The second cover layer can be the upper most layer of the dressing or can be a separate envelope that encloses the integrated components of the topical negative pressure system.

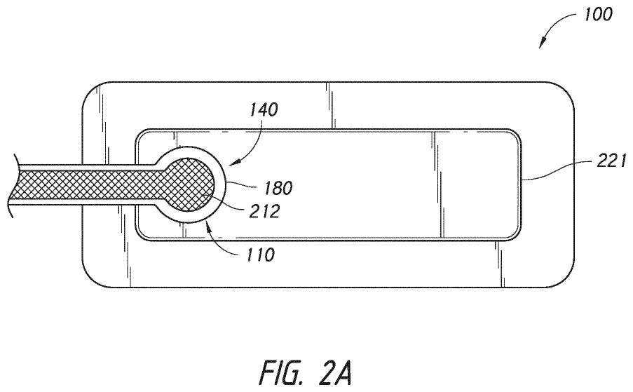

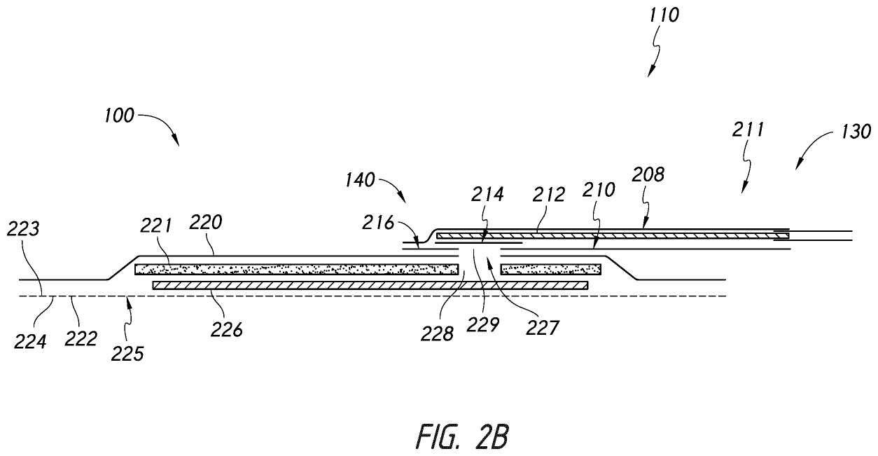

As shown in FIG. 2A, the fluidic connector 110 preferably comprises an enlarged distal end, or head 140 that is in fluidic communication with the dressing 100 as will be described in further detail below. In one embodiment, the enlarged distal end has a round or circular shape. The head 140 is illustrated here as being positioned near an edge of the dressing 100, but may also be positioned at any location on the dressing. For example, some embodiments may provide for a centrally or off-centered location not on or near an edge or corner of the dressing 100. In some embodiments, the dressing 100 may comprise two or more fluidic connectors 110, each comprising one or more heads 140, in fluidic communication therewith. In a preferred embodiment, the head 140 may measure 30 mm along its widest edge. The head 140 forms at least in part the applicator 180, described above, that is configured to seal against a top surface of the wound dressing.

FIG. 2B illustrates a cross-section through a wound dressing 100 similar to the wound dressing 100 as shown in FIG. 1B and described in International Patent Publication WO2013175306 A2, filed May 22, 2013, entitled “APPARATUSES AND METHODS FOR NEGATIVE PRESSURE WOUND THERAPY”, the disclosure of which is hereby incorporated by reference in its entirety, along with fluidic connector 110. The wound dressing 100, which can alternatively be any wound dressing embodiment disclosed herein or any combination of features of any number of wound dressing embodiments disclosed herein, can be located over a wound site to be treated. The dressing 100 may be placed as to form a sealed cavity over the wound site. In a preferred embodiment, the dressing 100 comprises a top or cover layer, or backing layer 220 attached to an optional wound contact layer 222, both of which are described in greater detail below. These two layers 220, 222 are preferably joined or sealed together so as to define an interior space or chamber. This interior space or chamber may comprise additional structures that may be adapted to distribute or transmit negative pressure, store wound exudate and other fluids removed from the wound, and other functions which will be explained in greater detail below. Examples of such structures, described below, include a transmission layer 226 and an absorbent layer 221.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound.

As illustrated in FIG. 2B, the wound contact layer 222 can be a polyurethane layer or polyethylene layer or other flexible layer which is perforated, for example via a hot pin process, laser ablation process, ultrasound process or in some other way or otherwise made permeable to liquid and gas. The wound contact layer 222 has a lower surface 224 and an upper surface 223. The perforations 225 preferably comprise through holes in the wound contact layer 222 which enable fluid to flow through the layer 222. The wound contact layer 222 helps prevent tissue ingrowth into the other material of the wound dressing. Preferably, the perforations are small enough to meet this requirement while still allowing fluid to flow therethrough. For example, perforations formed as slits or holes having a size ranging from 0.025 mm to 1.2 mm are considered small enough to help prevent tissue ingrowth into the wound dressing while allowing wound exudate to flow into the dressing. In some configurations, the wound contact layer 222 may help maintain the integrity of the entire dressing 100 while also creating an air tight seal around the absorbent pad in order to maintain negative pressure at the wound.

Some embodiments of the wound contact layer 222 may also act as a carrier for an optional lower and upper adhesive layer (not shown). For example, a lower pressure sensitive adhesive may be provided on the lower surface 224 of the wound dressing 100 whilst an upper pressure sensitive adhesive layer may be provided on the upper surface 223 of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized may be helpful to adhere the wound dressing 100 to the skin around a wound site. In some embodiments, the wound contact layer may comprise perforated polyurethane film. The lower surface of the film may be provided with a silicone pressure sensitive adhesive and the upper surface may be provided with an acrylic pressure sensitive adhesive, which may help the dressing maintain its integrity. In some embodiments, a polyurethane film layer may be provided with an adhesive layer on both its upper surface and lower surface, and all three layers may be perforated together.

A layer 226 of porous material can be located above the wound contact layer 222. This porous layer, or transmission layer, 226 allows transmission of fluid including liquid and gas away from a wound site into upper layers of the wound dressing. In particular, the transmission layer 226 preferably ensures that an open air channel can be maintained to communicate negative pressure over the wound area even when the absorbent layer has absorbed substantial amounts of exudates. The layer 226 should preferably remain open under the typical pressures that will be applied during negative pressure wound therapy as described above, so that the whole wound site sees an equalized negative pressure. The layer 226 may be formed of a material having a three dimensional structure. For example, a knitted or woven spacer fabric (for example Baltex 7970 weft knitted polyester) or a non-woven fabric could be used.

In some embodiments, the transmission layer 226 comprises a 3D polyester spacer fabric layer including a top layer (that is to say, a layer distal from the wound-bed in use) which is a 84/144 textured polyester, and a bottom layer (that is to say, a layer which lies proximate to the wound bed in use) which is a 10 denier flat polyester and a third layer formed sandwiched between these two layers which is a region defined by a knitted polyester viscose, cellulose or the like monofilament fiber. Other materials and other linear mass densities of fiber could of course be used.

Whilst reference is made throughout this disclosure to a monofilament fiber it will be appreciated that a multistrand alternative could of course be utilized. The top spacer fabric thus has more filaments in a yarn used to form it than the number of filaments making up the yarn used to form the bottom spacer fabric layer.

This differential between filament counts in the spaced apart layers helps control moisture flow across the transmission layer. Particularly, by having a filament count greater in the top layer, that is to say, the top layer is made from a yarn having more filaments than the yarn used in the bottom layer, liquid tends to be wicked along the top layer more than the bottom layer. In use, this differential tends to draw liquid away from the wound bed and into a central region of the dressing where the absorbent layer 221 helps lock the liquid away or itself wicks the liquid onwards towards the cover layer where it can be transpired.

Preferably, to improve the liquid flow across the transmission layer 226 (that is to say perpendicular to the channel region formed between the top and bottom spacer layers, the 3D fabric may be treated with a dry cleaning agent (such as, but not limited to, Perchloro Ethylene) to help remove any manufacturing products such as mineral oils, fats and/or waxes used previously which might interfere with the hydrophilic capabilities of the transmission layer. In some embodiments, an additional manufacturing step can subsequently be carried in which the 3D spacer fabric is washed in a hydrophilic agent (such as, but not limited to, Feran Ice 30 g/l available from the Rudolph Group). This process step helps ensure that the surface tension on the materials is so low that liquid such as water can enter the fabric as soon as it contacts the 3D knit fabric. This also aids in controlling the flow of the liquid insult component of any exudates.

A layer 221 of absorbent material is provided above the transmission layer 226. The absorbent material, which comprise a foam or non-woven natural or synthetic material, and which may optionally comprise a super-absorbent material, forms a reservoir for fluid, particularly liquid, removed from the wound site. In some embodiments, the layer 221 may also aid in drawing fluids towards the backing layer 220.

The material of the absorbent layer 221 may also prevent liquid collected in the wound dressing 100 from flowing freely within the dressing, and preferably acts so as to contain any liquid collected within the dressing. The absorbent layer 221 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 221 may typically be manufactured from ALLEVYN™ foam, Freudenberg 114-224-4 and/or Chem-Posite™ 11C-450. In some embodiments, the absorbent layer 221 may comprise a composite comprising superabsorbent powder, fibrous material such as cellulose, and bonding fibers. In a preferred embodiment, the composite is an airlaid, thermally-bonded composite.

In some embodiments, the absorbent layer 221 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. The wicking action also assists in bringing liquid into contact with the upper cover layer to aid increase transpiration rates of the dressing.

An aperture, hole, or orifice 227 is preferably provided in the backing layer 220 to allow a negative pressure to be applied to the dressing 100. The fluidic connector 110 is preferably attached or sealed to the top of the backing layer 220 over the orifice 227 made into the dressing 100, and communicates negative pressure through the orifice 227. A length of tubing may be coupled at a first end to the fluidic connector 110 and at a second end to a pump unit (not shown) to allow fluids to be pumped out of the dressing. Where the fluidic connector is adhered to the top layer of the wound dressing, a length of tubing may be coupled at a first end of the fluidic connector such that the tubing, or conduit, extends away from the fluidic connector parallel or substantially to the top surface of the dressing. The fluidic connector 110 may be adhered and sealed to the backing layer 220 using an adhesive such as an acrylic, cyanoacrylate, epoxy, UV curable or hot melt adhesive. The fluidic connector 110 may be formed from a soft polymer, for example a polyethylene, a polyvinyl chloride, a silicone or polyurethane having a hardness of 30 to 90 on the Shore A scale. In some embodiments, the fluidic connector 110 may be made from a soft or conformable material.

Preferably the absorbent layer 221 includes at least one through hole 228 located so as to underlie the fluidic connector 110. The through hole 228 may in some embodiments be the same size as the opening 227 in the backing layer, or may be bigger or smaller. As illustrated in FIG. 2B a single through hole can be used to produce an opening underlying the fluidic connector 110. It will be appreciated that multiple openings could alternatively be utilized. Additionally, should more than one port be utilized according to certain embodiments of the present disclosure one or multiple openings may be made in the absorbent layer and the obscuring layer in registration with each respective fluidic connector. Although not essential to certain embodiments of the present disclosure the use of through holes in the super-absorbent layer may provide a fluid flow pathway which remains unblocked in particular when the absorbent layer is near saturation.

The aperture or through-hole 228 is preferably provided in the absorbent layer 221 beneath the orifice 227 such that the orifice is connected directly to the transmission layer 226 as illustrated in FIG. 2B. This allows the negative pressure applied to the fluidic connector 110 to be communicated to the transmission layer 226 without passing through the absorbent layer 221. This ensures that the negative pressure applied to the wound site is not inhibited by the absorbent layer as it absorbs wound exudates. In other embodiments, no aperture may be provided in the absorbent layer 221, or alternatively a plurality of apertures underlying the orifice 227 may be provided. In further alternative embodiments, additional layers such as another transmission layer or an obscuring layer such as described in US Patent Publication 2015/0190286 A1, the entirety of which is hereby incorporated by reference, may be provided over the absorbent layer 221 and beneath the backing layer 220.

The backing layer 220 is preferably gas impermeable, but moisture vapor permeable, and can extend across the width of the wound dressing 100. The backing layer 220, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way, an effective chamber is made between the backing layer 220 and a wound site where a negative pressure can be established. The backing layer 220 is preferably sealed to the wound contact layer 222 in a border region around the circumference of the dressing, ensuring that no air is drawn in through the border area, for example via adhesive or welding techniques. The backing layer 220 protects the wound from external bacterial contamination (bacterial barrier) and allows liquid from wound exudates to be transferred through the layer and evaporated from the film outer surface. The backing layer 220 preferably comprises two layers; a polyurethane film and an adhesive pattern spread onto the film. The polyurethane film is preferably moisture vapor permeable and may be manufactured from a material that has an increased water transmission rate when wet. In some embodiments the moisture vapor permeability of the backing layer increases when the backing layer becomes wet. The moisture vapor permeability of the wet backing layer may be up to about ten times more than the moisture vapor permeability of the dry backing layer.

The absorbent layer 221 may be of a greater area than the transmission layer 226, such that the absorbent layer overlaps the edges of the transmission layer 226, thereby ensuring that the transmission layer does not contact the backing layer 220. This provides an outer channel of the absorbent layer 221 that is in direct contact with the wound contact layer 222, which aids more rapid absorption of exudates to the absorbent layer. Furthermore, this outer channel ensures that no liquid is able to pool around the circumference of the wound cavity, which could seep through the seal around the perimeter of the dressing leading to the formation of leaks. As illustrated in FIGS. 2A-2B, the absorbent layer 221 may define a smaller perimeter than that of the backing layer 220, such that a boundary or border region is defined between the edge of the absorbent layer 221 and the edge of the backing layer 220.

As shown in FIG. 2B, one embodiment of the wound dressing 100 comprises an aperture 228 in the absorbent layer 221 situated underneath the fluidic connector 110. In use, for example when negative pressure is applied to the dressing 100, a wound facing portion of the fluidic connector may thus come into contact with the transmission layer 226, which can thus aid in transmitting negative pressure to the wound site even when the absorbent layer 221 is filled with wound fluids. Some embodiments may have the backing layer 220 be at least partly adhered to the transmission layer 226. In some embodiments, the aperture 228 is at least 1-2 mm larger than the diameter of the wound facing portion of the fluidic connector 110, or the orifice 227.

In particular for embodiments with a single fluidic connector 110 and through hole, it may be preferable for the fluidic connector 110 and through hole to be located in an off-center position as illustrated in FIG. 2A. Such a location may permit the dressing 100 to be positioned onto a patient such that the fluidic connector 110 is raised in relation to the remainder of the dressing 100. So positioned, the fluidic connector 110 and the filter 214 (described below) may be less likely to come into contact with wound fluids that could prematurely occlude the filter 214 so as to impair the transmission of negative pressure to the wound site.

Turning now to the fluidic connector 110, preferred embodiments comprise a sealing surface 216, a bridge 211 (corresponding to bridge 120 in FIGS. 1A-1B) with a proximal end 130 and a distal end 140, and a filter 214. The sealing surface 216 preferably forms the applicator previously described that is sealed to the top surface of the wound dressing. In some embodiments, a bottom layer of the fluidic connector 110 may comprise the sealing surface 216. The fluidic connector 110 may further comprise an upper surface vertically spaced from the sealing surface 216, which in some embodiments is defined by a separate upper layer of the fluidic connector. In other embodiments, the upper surface and the lower surface may be formed from the same piece of material. In some embodiments, the sealing surface 216 may comprise at least one aperture 229 therein to communicate with the wound dressing. In some embodiments, the filter 214 may be positioned across the opening 229 in the sealing surface, and may span the entire opening 229. The sealing surface 216 may be configured for sealing the fluidic connector to the cover layer of the wound dressing, and may comprise an adhesive or weld. In some embodiments, the sealing surface 216 may be placed over an orifice in the cover layer. In other embodiments, the sealing surface 216 may be positioned over an orifice in the cover layer and an aperture in the absorbent layer 220, permitting the fluidic connector 110 to provide air flow through the transmission layer 226. In some embodiments, the bridge 211 may comprise a first fluid passage 212 in communication with a source of negative pressure, the first fluid passage 212 comprising a porous material, such as a 3D knitted material, which may be the same or different than the porous layer 226 described previously. The bridge 211 is preferably encapsulated by at least one flexible film layer 208, 210 having a proximal and distal end and configured to surround the first fluid passage 212, the distal end of the flexible film being connected to the sealing surface 216. The filter 214 is configured to substantially prevent wound exudate from entering the bridge.

Some embodiments may further comprise an optional second fluid passage positioned above the first fluid passage 212. For example, some embodiments may provide for an air leak may be disposed at the proximal end of the top layer 208 that is configured to provide an air path into the first fluid passage 212 and dressing 100 similar to the suction adapter as described in U.S. Pat. No 8,801,685, filed Dec. 30, 2011, entitled “APPARATUSES AND METHODS FOR NEGATIVE PRESSURE WOUND THERAPY” the disclosure of which is hereby incorporated by reference in its entirety.

Preferably, the fluid passage 212 is constructed from a compliant material that is flexible and that also permits fluid to pass through it if the spacer is kinked or folded over. Suitable materials for the fluid passage 212 include without limitation foams, including open-cell foams such as polyethylene or polyurethane foam, meshes, 3D knitted fabrics, non-woven materials, and fluid channels. In some embodiments, the fluid passage 212 may be constructed from materials similar to those described above in relation to the transmission layer 226. Advantageously, such materials used in the fluid passage 212 not only permit greater patient comfort, but may also provide greater kink resistance, such that the fluid passage 212 is still able to transfer fluid from the wound toward the source of negative pressure while being kinked or bent.

In some embodiments, the fluid passage 212 may be comprised of a wicking fabric, for example a knitted or woven spacer fabric (such as a knitted polyester 3D fabric, Baltex 7970®, or Gehring 879®) or a nonwoven fabric. These materials selected are preferably suited to channeling wound exudate away from the wound and for transmitting negative pressure and/or vented air to the wound site, and may also confer a degree of kinking or occlusion resistance to the fluid passage 212. In some embodiments, the wicking fabric may have a three-dimensional structure, which in some cases may aid in wicking fluid or transmitting negative pressure. In certain embodiments, including wicking fabrics, these materials remain open and capable of communicating negative pressure to a wound area under the typical pressures used in negative pressure therapy, for example between 40 to 150 mmHg. In some embodiments, the wicking fabric may comprise several layers of material stacked or layered over each other, which may in some cases be useful in preventing the fluid passage 212 from collapsing under the application of negative pressure. In other embodiments, the wicking fabric used in the fluid passage 212 may be between 1.5 mm and 6 mm; more preferably, the wicking fabric may be between 3 mm and 6 mm thick, and may be comprised of either one or several individual layers of wicking fabric. In other embodiments, the fluid passage 212 may be between 1.2-3 mm thick, and preferably thicker than 1.5 mm. Some embodiments, for example a suction adapter used with a dressing which retains liquid such as wound exudate, may employ hydrophobic layers in the fluid passage 212, and only gases may travel through the fluid passage 212. Additionally, and as described previously, the materials used in the system are preferably conformable and soft, which may help to avoid pressure ulcers and other complications which may result from a wound treatment system being pressed against the skin of a patient.

Preferably, the filter element 214 is impermeable to liquids, but permeable to gases, and is provided to act as a liquid barrier and to ensure that no liquids are able to escape from the wound dressing 100. The filter element 214 may also function as a bacterial barrier. Typically the pore size is 0.2 nm. Suitable materials for the filter material of the filter element 214 include 0.2 micron Gore™ expanded PTFE from the MMT range, PALL Versapore™ 200R, and Donaldson™ TX6628. Larger pore sizes can also be used but these may require a secondary filter layer to ensure full bioburden containment. As wound fluid contains lipids it is preferable, though not essential, to use an oleophobic filter membrane for example 1.0 micron MMT-332 prior to 0.2 micron MMT-323. This prevents the lipids from blocking the hydrophobic filter. The filter element can be attached or sealed to the port and/or the cover film over the orifice. For example, the filter element 214 may be molded into the fluidic connector 110, or may be adhered to one or both of the top of the cover layer and bottom of the suction adapter 110 using an adhesive such as, but not limited to, a UV cured adhesive.

It will be understood that other types of material could be used for the filter element 214. More generally a microporous membrane can be used which is a thin, flat sheet of polymeric material, this contains billions of microscopic pores. Depending upon the membrane chosen these pores can range in size from 0.01 to more than 10 micrometers. Microporous membranes are available in both hydrophilic (water filtering) and hydrophobic (water repellent) forms. In some embodiments of the invention, filter element 214 comprises a support layer and an acrylic co-polymer membrane formed on the support layer. Preferably the wound dressing 100 according to certain embodiments of the present invention uses microporous hydrophobic membranes (MHMs). Numerous polymers may be employed to form MHMs. For example, the MHMs may be formed from one or more of PTFE, polypropylene, PVDF and acrylic copolymer. All of these optional polymers can be treated in order to obtain specific surface characteristics that can be both hydrophobic and oleophobic. As such these will repel liquids with low surface tensions such as multi-vitamin infusions, lipids, surfactants, oils and organic solvents.

MHMs block liquids whilst allowing air to flow through the membranes. They are also highly efficient air filters eliminating potentially infectious aerosols and particles. A single piece of MHM is well known as an option to replace mechanical valves or vents. Incorporation of MHMs can thus reduce product assembly costs improving profits and costs/benefit ratio to a patient.

The filter element 214 may also include an odor absorbent material, for example activated charcoal, carbon fiber cloth or Vitec Carbotec-RT Q2003073 foam, or the like. For example, an odor absorbent material may form a layer of the filter element 214 or may be sandwiched between microporous hydrophobic membranes within the filter element. The filter element 214 thus enables gas to be exhausted through the orifice. Liquid, particulates and pathogens however are contained in the dressing.

Similar to the embodiments of wound dressings described above, some wound dressings comprise a perforated wound contact layer with silicone adhesive on the skin-contact face and acrylic adhesive on the reverse. Above this bordered layer sits a transmission layer or a 3D spacer fabric pad. Above the transmission layer, sits an absorbent layer. The absorbent layer can include a superabsorbent non-woven (NW) pad. The absorbent layer can over-border the transmission layer by approximately 5 mm at the perimeter. The absorbent layer can have an aperture or through-hole toward one end. The aperture can be about 10 mm in diameter. Over the transmission layer and absorbent layer lies a backing layer. The backing layer can be a high moisture vapor transmission rate (MVTR) film, pattern coated with acrylic adhesive. The high MVTR film and wound contact layer encapsulate the transmission layer and absorbent layer, creating a perimeter border of approximately 20 mm. The backing layer can have a 10 mm aperture that overlies the aperture in the absorbent layer. Above the hole can be bonded a fluidic connector that comprises a liquid-impermeable, gas-permeable semi-permeable membrane (SPM) or filter that overlies the aforementioned apertures.

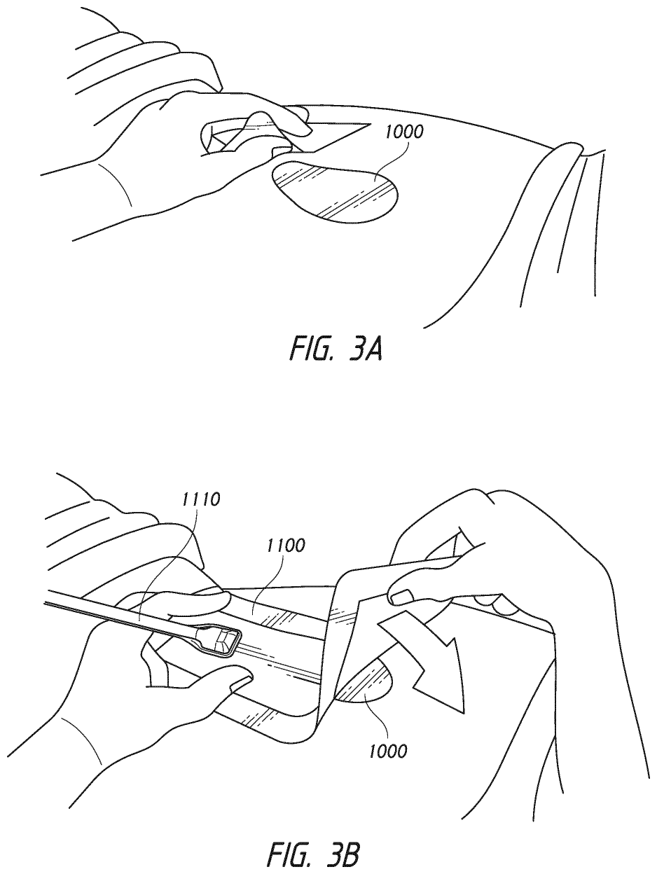

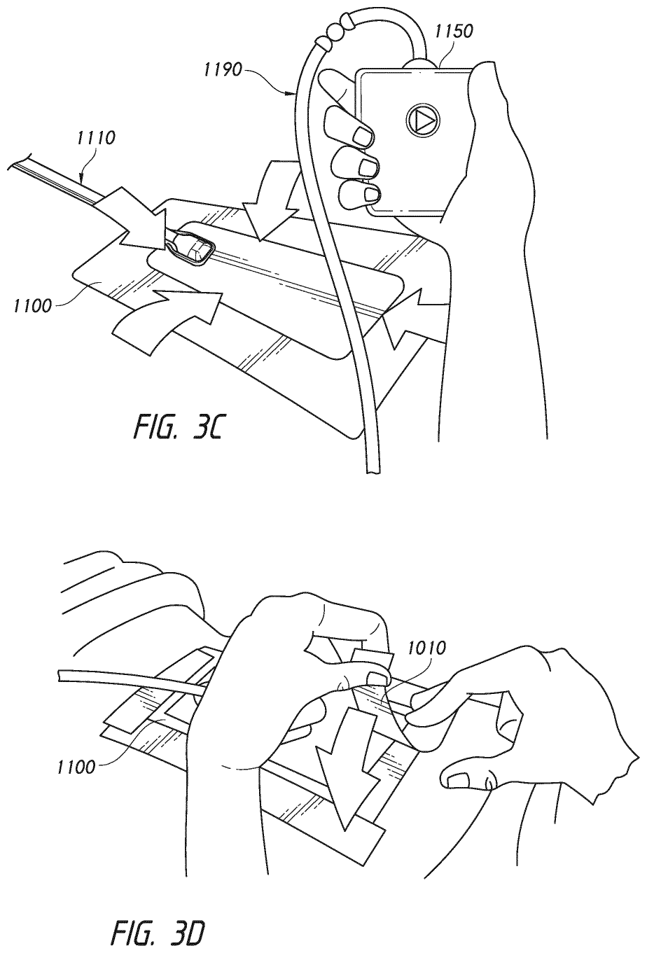

FIGS. 3A-D illustrate the use of an embodiment of a negative pressure therapy wound treatment system being used to treat a wound site on a patient. FIG. 3A shows a wound site 1000 being cleaned and prepared for treatment. Here, the healthy skin surrounding the wound site 1000 is preferably cleaned and excess hair removed or shaved. The wound site 1000 may also be irrigated with sterile saline solution if necessary. Optionally, a skin protectant may be applied to the skin surrounding the wound site 1000. If necessary, a wound packing material, such as foam or gauze, may be placed in the wound site 1000. This may be preferable if the wound site 1000 is a deeper wound.

After the skin surrounding the wound site 1000 is dry, and with reference now to FIG. 3B, the wound dressing 1100 may be positioned and placed over the wound site 1000. Preferably, the wound dressing 1100 is placed with the wound contact layer over and/or in contact with the wound site 1000. In some embodiments, an adhesive layer is provided on the lower surface of the wound contact layer, which may in some cases be protected by an optional release layer to be removed prior to placement of the wound dressing 1100 over the wound site 1000. Preferably, the dressing 1100 is positioned such that the fluidic connector 1110 is in a raised position with respect to the remainder of the dressing 1100 so as to avoid fluid pooling around the port. In some embodiments, the dressing 1100 is positioned so that the fluidic connector 1110 is not directly overlying the wound, and is level with or at a higher point than the wound. To help ensure adequate sealing for TNP, the edges of the dressing 1100 are preferably smoothed over to avoid creases or folds.

With reference now to FIG. 3C, the dressing 1100 is connected to the pump 1150. The pump 1150 is configured to apply negative pressure to the wound site via the dressing 1100, and typically through a conduit. In some embodiments, and as described herein, a fluidic connector 1110 may be used to join the conduit 1190 from the pump to the dressing 1100. Where the fluidic connector is adhered to the top layer of the wound dressing, a length of tubing may be coupled at a first end of the fluidic connector such that the tubing, or conduit, extends away from the fluidic connector parallel to the top of the dressing. In some embodiments, the conduit may comprise a fluidic connector. It is expressly contemplated that a conduit may be a soft bridge, a hard tube, or any other apparatus which may serve to transport fluid. Upon the application of negative pressure with the pump 1150, the dressing 1100 may in some embodiments partially collapse and present a wrinkled appearance as a result of the evacuation of some or all of the air underneath the dressing 1100. In some embodiments, the pump 1150 may be configured to detect if any leaks are present in the dressing 1100, such as at the interface between the dressing 1100 and the skin surrounding the wound site 1000. Should a leak be found, such leak is preferably remedied prior to continuing treatment.

Turning to FIG. 3D, additional fixation strips 1010 may also be attached around the edges of the dressing 1100. Such fixation strips 1010 may be advantageous in some situations so as to provide additional sealing against the skin of the patient surrounding the wound site 1000. For example, the fixation strips 1010 may provide additional sealing for when a patient is more mobile. In some cases, the fixation strips 1010 may be used prior to activation of the pump 1150, particularly if the dressing 1100 is placed over a difficult to reach or contoured area.

Treatment of the wound site 1000 preferably continues until the wound has reached a desired level of healing. In some embodiments, it may be desirable to replace the dressing 1100 after a certain time period has elapsed, or if the dressing is full of wound fluids. During such changes, the pump 1150 may be kept, with just the dressing 1100 being changed.

### Absorbent Materials Within the Wound Dressing

In certain embodiments, such as described above in relation to FIG. 2B, fluid (for example, wound exudate) is handled by the dressing 100 by passing through the perforated wound contact layer 222, into the transmission layer 226, and is then absorbed and retained by the absorbent layer 221. Fluid is then able to evaporate through the breathable backing layer 220. However, in some embodiments, wound dressings such as those described above can have complicated structure and can require complex manufacturing processes.

The terms regions, areas, and/or portions of absorbent material refer to the portions of material that make up the absorbent layer and these terms can be used interchangeably herein and throughout the specification. In some embodiments, the portions of absorbent material can be separated by a space between them and can be formed into one integral piece. Regions, areas, or portions that are separated by space between them, may however touch or come into contact with one another. For example, two regions, areas, or portions can be separated by a space or other region of material between them within an absorbent layer but can be integrally formed or substantially connected together.

In a negative pressure wound dressing a key performance requirement is that air can flow freely through the dressing to enable the wound dressing to deliver a negative air pressure to the wound. This restricts the use of super-absorbent polymers, which though they provide a high level of absorbency, once they have absorbed wound exudate they swell in a way which causes them to agglomerate and close any pores in the system which air may flow through to maintain the negative pressure. This behavior of the super-absorber is termed as ‘gel-blocking’ and has been observed when using superabsorbent materials including superabsorbent particles and fibers.

Two of the key performance parameters required in a negative pressure dressing are: (1) high exudate absorbency; and (2) free flow of air through dressing to enable negative pressure to be maintained

The use of superabsorbent materials is a benefit as they enable high exudate absorbency. However, in some configurations, their ‘gel-blocking’ nature restricts the free flow of air in the dressing. To address this, in some embodiments, multi-layer wound dressing systems are used similar to the multi-layer wound dressing systems described with reference to FIGS. 2A-2B. One layer of the dressing maintains the air flow (spacer or transmission layer) whilst another layer contains absorbent material (for example, superabsorbent particles) and provides the absorbency function (absorbent layer).

As described herein, the spacer or transmission layer can be a 3D knitted textile. Due to the complex construction and manufacturing process, the 3D knitted textile carries a high cost to manufacture in comparison to other materials used in wound dressing constructions. However, it's unique structure enables it to have performance advantages over 2D textile substrates.

Currently available options for providing super-absorbers into wound dressings presents the super-absorber as being homogenously distributed, i.e. the superabsorbent particles are evenly distributed throughout the substrate. This is also the case with nonwoven materials containing superabsorbent fibers. Current traditional nonwovens processing results in the fibers being randomly distributed throughout the substrate with no control over placement.

### Open Pore Absorbent Layer

In some embodiments, the absorbent layer can include superabsorbent materials as a structural parameter in the dressing. The superabsorbent materials can be used in a targeted manner which, as it swells, becomes a ‘scaffolding’ or gives the dressing structural integrity, opening pores which air is able to flow through. This can utilize what is considered as a negative behavior of the superabsorbent materials, its ability to swell and ‘gel block’, as a structural parameter in the dressing. In some embodiments, the approach can enable a high level of exudate or other fluids to be absorbed. As the exudate is absorbed the pores in the dressing can open further, allowing the air to flow and thus negative pressure to be delivered.

Using two separate material layers as described with reference to FIGS. 2A-2B to provide two separate function layers can carry cost in both the raw materials, e.g. transport, compliance checking of two goods, and in processing and converting, e.g. manufacturing time and cost associated with processing and joining two separate materials. Also, the spacer or transmission layer, due to its complex construction and manufacturing technique, can be a high cost raw material. Therefore, it can be useful to provide the advantages of the spacer or transmission layer within the absorbent material layer.

The overall advantages involved with switching to a single layer system to provide air flow and absorbent properties such as described herein can include: (1) Cost saving on raw materials (replacing expensive knitted spacer layer); (2) Cost saving on processing (processing one layer instead of two); and (3) Potential enhanced performance in absorbency.

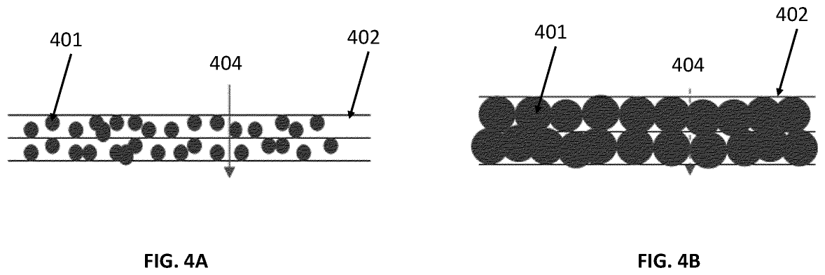

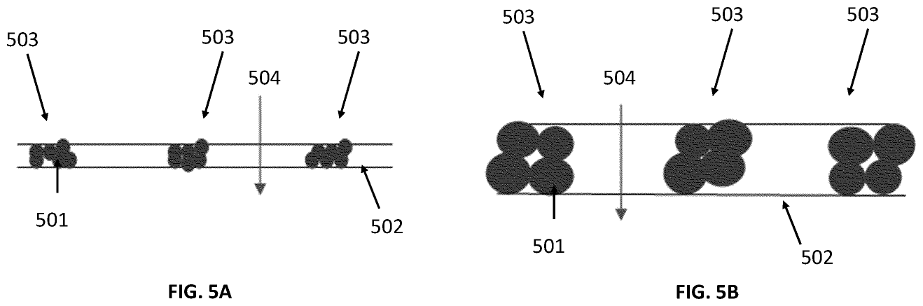

The superabsorbent material within the absorbent layer can be positioned to provide the flow of air. As the exudate is absorbed the pores in the dressing can open further, allowing the air to flow and thus negative pressure to be delivered. FIGS. 4A-5B illustrate embodiments of superabsorbent particles within a dressing layer.

FIG. 4A illustrates an embodiment of dry superabsorbent particles distributed amongst material layers of the dressing. As illustrated in FIG. 4A, the dry superabsorbent particles 401 can be distributed randomly within the material layer 402. In some embodiments, the material layer 402 can be a nonwoven layer or any other layer of material described herein. This configuration of the superabsorbent particles 401 can be similar to traditional placement of superabsorbent particles within material layers used in would dressings. FIG. 4B illustrates an embodiment of wet superabsorbent particles 401 in the material layer 402. As illustrated in FIG. 4B, the wet particles can swell and agglomerate together thereby restricting the air flow (shown by arrow 404 passing through the material layer in FIGS. 4A-4B).

FIG. 5A illustrates an embodiment of a targeted application of superabsorbent particles. As illustrated in FIG. 5A, the superabsorbent particles 501 can be arranged in targeted areas 503 of the material layer 502. In some embodiments, the material layer 502 can be an absorbent layer within a wound dressing system as described herein. In some embodiments, the material layer 502 can be a nonwoven layer or any other layer of material described herein. The superabsorbent particles 501 can be arranged in discrete regions or targeted areas 503 within the material layer 502 to allow an air path 504 (shown by arrow passing through the material layer) to allow air to flow through the material layer 502. In some embodiments, the superabsorbent particles can be arranged in a pattern or randomly spaced while maintaining a space for the air path between the regions of the superabsorbent particles for continued air flow through the material layer once the superabsorbent particles swell.

FIG. 5B illustrates an embodiment of wet superabsorbent particles 501 in a targeted application within a wound dressing layer 502. As illustrated in FIG. 5B, when the superabsorbent particles 501 are wetted, due to the targeted application of superabsorbent particles 501, the air path 504 within the dressing layer 502 can be maintained. In some embodiments, superabsorbent particles can be aggregated in distinct spaced apart locations from other aggregations of superabsorbent particles. The aggregations of superabsorbent particles can be distributed randomly or in a pattern across the absorbent layer. In some embodiments, the absorbent layer can include a plurality of target areas of superabsorbent material. The plurality of target areas of superabsorbent material can be arranged in various patterns including a spaced pattern and/or a regularly repeating pattern, such as a grid. Although FIGS. 5A-5B illustrated the targeted areas and application of superabsorbent particles, other superabsorbent materials can be used to create the targeted areas of superabsorbent material within the absorbent layer and/or spacer layer including but not limited to super absorbent yarn and superabsorbent fibers.

In some embodiments, the dressing with superabsorbent material can utilizes a superabsorbent yarn in an open net structure. As the net structure is very open, even when the yarn is swollen to maximum capacity there is still an air path.

In one embodiment, the yarn can be stabilized between two melt spun nonwovens which provide some stability but only limited absorbency. In other embodiments, the nonwovens with limited absorbency can be substituted for more absorbent materials such as viscose based hydroentangled or needlebonded nonwovens.

The superabsorbent material can be applied in a targeted or zoned manner in a variety of formats and methods.

In some embodiments, the dressing with superabsorbent material can utilizes superabsorbent particles. In some embodiments, the superabsorbent particles can be applied in a screen printed manner.

In some embodiments, a glue configuration can be applied to the substrate. The substrate is then covered with superabsorbent particles before compressed air is used to remove the unsecured superabsorbent particles. This process can enable targeted application of the superabsorbent particles.

In some embodiments, the superabsorbent particles can be introduced into 3D structures created in a nonwoven structure (i.e. NIRI Hydrospace) or be injected into an engineered 3D knitted spacer or transmission layer.

In some embodiments, the dressing with superabsorbent material can utilizes superabsorbent fibers. A web or nonwoven of superabsorbent fibers can be point bonded onto another nonwoven substrate to create a zoned area of high absorbency and the excess removed. For example, a distinct pattern can be cut out then the excess can be removed. In some embodiments, if u-bonded fibers are used these may be re-carded and recycled.

In some embodiments, a web of superabsorbent fibers can be stitch bonded or sewn in a manner that restricts their ability to swell when wetted. This configuration can create air panels in the structure (for example, such as a quilting affect).

In some embodiments, the dressing with superabsorbent material can utilizes superabsorbent yarn. Yarn may be used in a net or other open structure. The yarn can be used in an open structure such as knitted or woven. For example, the yarn in a woven or knitted structure may be integrated into a nonwoven web via needling, through air oven bonding, calendaring or laminating.

In some embodiments, the absorbent material layer with the targeted application of superabsorbent material can be positioned within a wound dressing similar to the wound dressing described with reference to FIGS. 2A-2B. In some embodiments, the wound dressing can include a single unit would dressing with the absorbent material layer enclosed within a wound contact layer and a backing or cover layer as described with reference to FIGS. 2A-2B. However, in some embodiments, due to the targeted application of superabsorbent material within the absorbent material layer, the spacer or transmission layer is optional. The dressing with an absorbent layer with the targeted application of superabsorbent material can comprise a backing layer attached to a wound contact layer, both of which are described in greater detail herein with reference to FIGS. 2A-2B. The wound contact layer can be configured to be placed in contact with the wound and the absorbent layer with the targeted application of superabsorbent material can be enclosed within the wound contact layer and cover layer. As described herein, the wound contact layer can be formed from a material configured to be removed from the wound with the other wound dressing layers.

In some embodiments, the superabsorbent material can be fibers (for example, Aquacel or Durafiber) or particles (e.g., sodium polyacrylate). In some embodiments, the targeted application of superabsorbent material can be separated by non-super absorbing materials or particles. In some embodiments, one or more portions of a wicking or acquisition distribution layer materials (ADL material), as described in U.S. patent application Ser. No. 14/418,908 filed Jan. 30, 2015, published as US 2015/0190286 A1 on Jul. 9, 2015, titled “WOUND DRESSING AND METHOD OF TREATMENT,” the disclosure of which is hereby incorporated by reference in its entireties, can be used between superabsorbent material regions. In some embodiments, one or more portions or areas of ADL materials can be disposed between targeted areas of superabsorbent materials within the absorbent layer. In some embodiments, the ADL material can horizontally wick fluid such as wound exudate as it is absorbed upward through the layers of the dressing. Lateral wicking of fluid may allow maximum distribution of the fluid through the absorbent layer and may enable the absorbent layer to reach its full holding capacity. This may advantageously increase moisture vapor permeation and efficient delivery of negative pressure to the wound site. Some embodiments of the ADL material may comprise viscose, polyester, polypropylene, cellulose, or a combination of some or all of these, and the material may be needle-punched. The ADL materials may be constructed from a material which resists compression under the levels of negative pressure commonly applied during negative pressure therapy.

In some embodiments, one or more layers of the absorbent layer with the targeted application of superabsorbent material can be used. For example, multiple layers of absorbent layers with the targeted application of superabsorbent material can be used. In some embodiments, the regions or areas of targeted application of superabsorbent material within a first material layer can be offset from regions or areas of targeted application of superabsorbent material within a second material layer. In other embodiments, the regions or areas of targeted application of superabsorbent material in a first layer can be stacked or partially overlapping the regions or areas of targeted application of superabsorbent material in a second layer.

In some embodiments, a wound dressing with the absorbent layer with the targeted areas of superabsorbent material can be positioned over a wound. The wound dressing can be configured to be used in combination with a source of negative pressure. The source of negative pressure can be in fluid communication with a wound site through the wound dressing and negative pressure can be maintained below the cover layer of the wound dressing and applied to the wound. As negative pressure is applied, fluids can be moved through the perforations in the wound contact layer and absorbed in the targeted areas of the superabsorbent material within the absorbent layer of the wound dressing. As fluids are absorbed within the targeted areas of the absorbent layer, an air flow path is maintained in the spaces between the superabsorbent material positioned at the one or more target areas of the absorbent layer.

When the dressing is positioned over the wound, the absorbent layer is positioned over the wound and wound fluid can pass through the wound contact layer into the absorbent layer and air flow (including air flow from the negative pressure source) can pass between the targeted areas of superabsorbent material in the absorbent layer. In this way, the targeted areas of superabsorbent material can allow for fluid to be absorbed by the superabsorbent material while maintaining an air path for air to flow through the absorbent layer.

In some embodiments, a source of negative pressure (such as a pump) and some or all other components of the topical negative pressure system, such as power source(s), sensor(s), connector(s), user interface component(s) (such as button(s), switch(es), speaker(s), screen(s), etc.) and the like, can be integral with the wound dressing. In some embodiments, the components can be integrated below, within, on top of, and/or adjacent to the backing layer. In some embodiments, the wound dressing can include a second cover layer and/or a second filter layer for positioning over the layers of the wound dressing and any of the integrated components. The second cover layer can be the upper most layer of the dressing or can be a separate envelope that encloses the integrated components of the topical negative pressure system.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A wound treatment apparatus comprising:
a wound dressing configured to be positioned over a wound site, the wound dressing comprising:
an absorbent layer for absorbing wound exudate, the absorbent layer comprising a superabsorbent material, wherein the superabsorbent material is positioned at one or more target areas of the absorbent layer;
a backing layer over the absorbent layer and comprising at least one orifice.

2. The wound treatment apparatus of claim 1, wherein the one or more target areas are positioned to allow an air path within the absorbent layer between or around the superabsorbent material in the one or more target areas when the superabsorbent materials are wet.

3. The wound treatment apparatus of claim 1, wherein the superabsorbent material is positioned within the absorbent layer such that when exudate is absorbed by the superabsorbent material, pores within the absorbent layer are opened to allow for flow of air.

4. The wound treatment apparatus of claim 1, wherein the superabsorbent material is screen printed to position the superabsorbent material at the one or more target areas of the absorbent layer.

5. The wound treatment apparatus of claim 1, wherein the superabsorbent material is glued to the absorbent layer to position the superabsorbent material at the one or more target areas of the absorbent layer.

6. The wound treatment apparatus of claim 1, wherein the absorbent layer is a 3D structure, wherein the superabsorbent material is injected into the 3D structure to position the superabsorbent material at the one or more target areas.

7. The wound treatment apparatus of claim 1, wherein the absorbent layer comprises a first nonwoven layer and the superabsorbent material comprises a second nonwoven layer of superabsorbent fibers, wherein the superabsorbent fibers are bonded onto the first nonwoven to position the superabsorbent material at the one or more target areas.

8. The wound treatment apparatus of claim 1, wherein the superabsorbent material comprises a web of superabsorbent fibers, wherein the superabsorbent fibers are stitch bonded, wherein the stitch bonding of the superabsorbent fibers is configured to restrict the ability of the superabsorbent fibers from swelling when wetted and create air panels in the absorbent layer.

9. The wound treatment apparatus of claim 1, wherein the superabsorbent material comprises a superabsorbent yarn, wherein the superabsorbent yam is integrated into the absorbent layer.

10. The wound treatment apparatus of claim 9, wherein the absorbent layer comprises a nonwoven web and the superabsorbent yarn is integrated into the nonwoven web.

11. The wound treatment apparatus of claim 1, further comprising a first transmission layer beneath the absorbent layer.

12. The wound treatment apparatus of claim 1, wherein the absorbent layer comprises a plurality of target areas of superabsorbent material.

13. The wound treatment apparatus of claim 12, wherein the plurality of target areas of superabsorbent material are arranged in a regularly repeating pattern.

14. The wound treatment apparatus of claim 12, wherein the plurality of target areas of superabsorbent material are arranged in a grid pattern.

15. The wound treatment apparatus of claim 12, wherein the plurality of target areas of superabsorbent material comprise a plurality of aggregated superabsorbent particles, each aggregation being spaced apart from one another.

16. The wound treatment apparatus of claim 1, further comprising a wound contact layer beneath the absorbent layer and sealed to the backing layer.

17. The wound treatment apparatus of claim 1, further comprising a wound contact layer beneath the absorbent layer and sealed to the backing layer, and without providing a separate transmission layer between the wound contact layer and the backing layer.

18. The wound treatment apparatus of claim 1, further comprising a source of negative pressure configured to be in fluid communication with a wound site through the wound dressing.

19. The wound treatment apparatus of claim 1, wherein a fluidic connector is positioned over the at least one orifice configured to provide negative pressure through the wound dressing to the wound site.

20. The wound treatment apparatus of claim 19, wherein the fluidic connector comprises a filter.

21. (canceled)

22. (canceled)
